(19) United States
(12) Patent Application Publication
(10) Pub. No.: US 2005/0060004 A1
(43) Pub. Date: Mar. 17, 2005

(54) Expanded ultra-high molecular weight polyethylene in an electrical medical device

(76) Inventors: Daniel J. Cooke, Lake Jackson, TX (US); Eric Hammill, Lauderdale, MN (US); Mohan Krishnan, Shoreview, MN (US)

Correspondence Address: Schwegman, Lundberg, Woessner & Kluth, P.A., P.O. Box 2938, Minneapolis, MN, 55402, US

(21) Appl. No.: 10/662,129

(22) Filed: Sep. 12, 2003

(51) Int. Cl. A61N 1/375

(52) U.S. Cl. 607/36

(57) ABSTRACT

An electro-medical system includes a porous covering that has a pore structure to resist in vivo fibrotic tissue ingrowth. The system includes an electrode where the porous covering includes a porous communication through the covering to the electrode. The system includes a can that is covered with the porous covering. The system also includes a can with a dielectric coating over the can, and the porous covering allows for a hot can. The system also includes a lead with a dielectric coating over the lead.

### TECHNICAL FIELD

The technical field relates to electro-medical devices. More particularly, an embodiment relates to a covering on an electro-medical device. In particular, an embodiment relates to a specialized polyethylene material that is formed on an electro-medical device.

### BACKGROUND

Electrical medical devices that are deployed in vivo are subject to various environmental influences within the patient. Examples of electrical medical devices include cardiac pacemakers and neurostimulators. An electrical source is housed in a container that is often referred to as a can. An electrical lead connects to the can and terminates in a target tissue region.

In the case of a cardiac stimulator such as a pacemaker or a defibrilator, excess lead is usually coiled or bunched in the tissue region that envelopes the can. The coiled or bunched configuration, allows for ordinary movement of the patient. During ordinary movement of the patient, the can slides over the coiled or bunched lead, and the coiled or bunched lead slides over itself. After a prolonged deployment in a patient, the extended service of the can and the lead may cause malfunctions, caused by abrasion of the lead.

Another challenge with conventional electrical medical devices includes ingrowth of fibrotic tissue around specific sites on the lead. Fibrotic tissue ingrowth can lead to clinical complications if the lead is extracted from the patient, and otherwise.

A further challenge includes leads that require sufficient axial strength while remaining flexible and pliable.

### SUMMARY

At least some of the above mentioned problems and challenges are overcome by embodiments set forth in this disclosure. An embodiment relates to an electro-medical system that includes a container. The container has a porous first covering, and porous first covering includes a porous communication to the container.

An embodiment includes the porous first covering of expanded ultra-high molecular weight polyethylene. An embodiment includes a lead that is coupled to the container. The lead is covered at an electrode portion with a porous second covering.

Another embodiment includes an electro-medical system container that is a lead, including a lead proximal end, a lead body, and a distal end including an electrode. At least the electrode includes a porous second covering that includes a porous communication to the electrode on the lead. The porous second covering includes a pore structure that repels in vivo fibrotic tissue ingrowth.

In another embodiment, the porous second covering extends a majority of the length of the lead from the lead proximal end to the lead distal end.

In anther embodiment, any porous structures used to contain the electrical device in the container, is expanded ultra-high molecular weight polyethylene.

In an embodiment, the porous second covering includes expanded ultra-high molecular weight polyethylene. In another embodiment, the lead is coupled to a container, and the container is covered with a porous first covering.

Another embodiment includes an electro-medical system that includes a can including a pulse generator in the can. A dielectric coating is disposed over the can. A passage exists through the dielectric coating to form an exposed portion of the can, but the porous first covering is over the exposed portion of the can.

Another embodiment relates to a pulse generator. The pulse generator includes a can, and in the can, at least a battery, a capacitor, and circuitry on a substrate that is used to deliver at least a ventricular contraction pulse. Between the battery and the circuitry on a substrate, there is at least one barrier of expanded ultra-high molecular weight polyethylene.

In another embodiment, the can contains a capacitor. The capacitor can also be isolated with at least one barrier of expanded ultra-high molecular weight polyethylene. In another embodiment, the can is coupled to a lead, and at least one of the lead and the can are covered with a porous covering that has a pore structure that repels in vivo fibrotic tissue ingrowth.

### BRIEF DESCRIPTION OF THE DRAWINGS

In order to illustrate the manner in which embodiments are obtained, a more particular description will be rendered by reference to specific embodiments thereof that are illustrated in the appended drawings. These drawings depict only typical embodiments that are not necessarily drawn to scale and are not to be considered to be limiting of its scope. The embodiments will be described and explained with additional specificity and detail through the use of the accompanying figures in which:

FIG. 1 is an elevation of a pulse generator can according to an embodiment;

FIG. 2 is a detail section of the pulse generator can, depicted in FIG. 1 that further illustrates an embodiment;

FIG. 3 is an elevation of a pulse generator can according to an embodiment;

FIG. 4 is an elevation of a pulse generator can according to an embodiment;

FIG. 5 is a schematic elevation of a lead according to an embodiment;

FIG. 6 is a detail section of the lead depicted in FIG. 5 that further illustrates an embodiment;

FIG. 7 is a cut-away elevation of a lead according to an embodiment;

FIG. 8 is a detail section of the lead depicted in FIG. 7 that further illustrates an embodiment;

FIG. 9 is a side cross-section of a lead according to an embodiment;

FIG. 10 is a detail section of the lead depicted in FIG. 7 that further illustrates an embodiment;

FIG. 11 is an elevation of a pulse generator can and a lead assembly according to an embodiment;

FIG. 12 is an elevation of a pulse generator can and a lead assembly according to an embodiment; and

FIG. 13 is a cut-away elevation of a pulse generator can according to an embodiment.

### DETAILED DESCRIPTION

In the following detailed description, reference is made to the accompanying drawings that form a part hereof, and in which is shown, by way of illustration, specific ways that embodiments may be practiced. In the drawings, like numerals describe substantially similar components throughout the several views. These embodiments are in sufficient detail to enable those skilled in the art to practice various embodiments. Other embodiments may be utilized and structural, logical, and electrical changes may be made without departing from the scope of the various embodiments.

The following description includes terms, such as upper, lower, first, second, etc. that are used for descriptive purposes only and are not to be construed as limiting. The embodiments of apparatus or article embodiments described herein can be manufactured, used, or shipped in a number of positions and orientations.

In the following illustrations, containers are depicted. In an embodiment, a container relates to a “can”, such as is variously illustrated in FIGS. 1-5 and elsewhere. In an embodiment, the “can” is a housing such as the housing for a pacemaker. In an embodiment, the “can” is a neurostimulator housing. In an embodiment, the “can” is a defibrillator housing. In an embodiment, the “can” is a monitor housing. In an embodiment, the “can” is a housing for a blood pressure monitor. In an embodiment, the “can” is a housing for a temperature monitor. In an embodiment, the “can” is a housing for a blood pressure monitor. In an embodiment, the “can” is a housing for a blood gas monitor, such as for dissolved oxygen, dissolved nitrogen, and other blood gases. In an embodiment, the “can” is a housing for a blood sugar monitor. In an embodiment, the “can” is a housing for a monitor for at least one lipoprotein. In an embodiment, the “can” is a housing an insulin monitor. In an embodiment, the “can” is a housing for an electrolyte monitor. In an embodiment, the “can” is a housing for a pulse monitor. In an embodiment, the “can” is a housing for a respiration monitor. In various embodiments in this disclosure, the “can” is referred to in relation to a cardiac pacemaker or a defibrilator. Other embodiments include the structural elements illustrated and discussed, but the “can” is a housing for each of the given functionalities and their combinations set forth in this disclosure. In an embodiment, the “can” is a housing for a monitor and/or a pulse generator that includes more than one of the above functionalities.

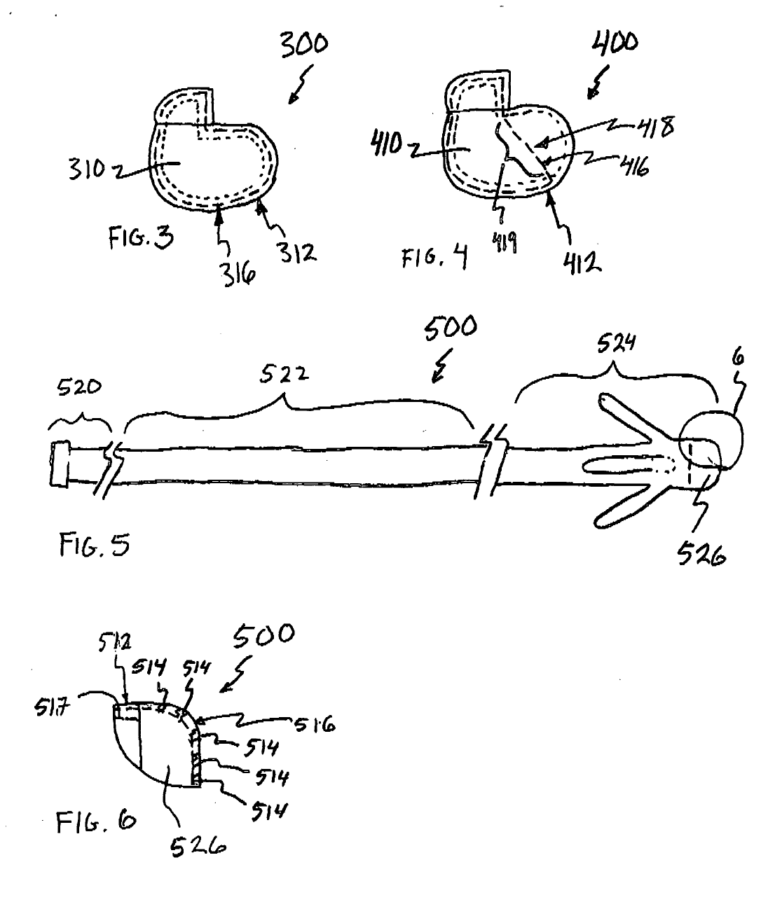

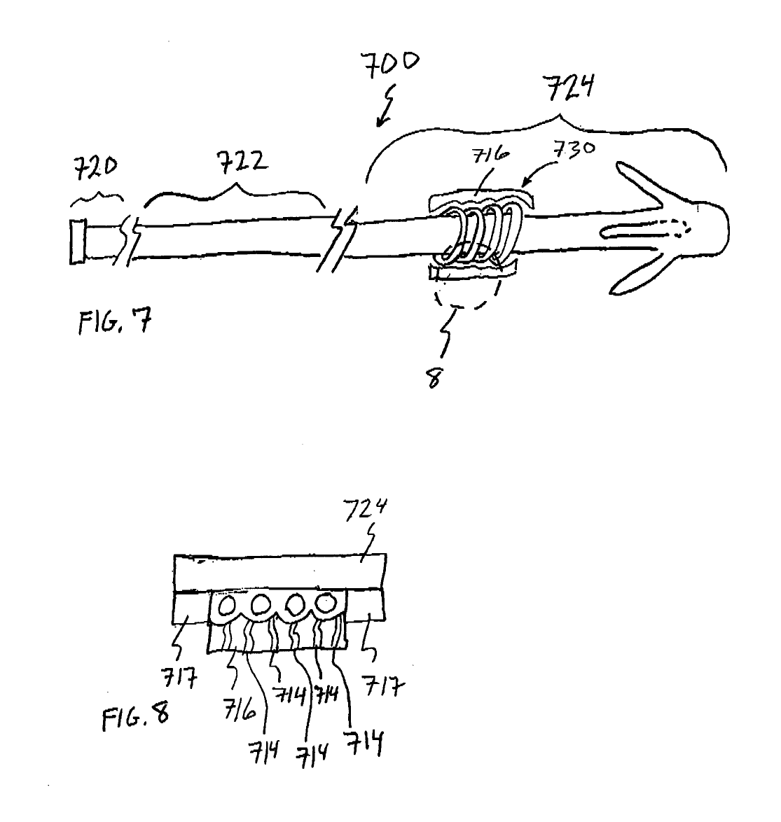

In an embodiment, the container is an electrode such as is illustrated and discussed in FIGS. 5-12 and elsewhere in this disclosure. In an embodiment, the container houses a wire. In an embodiment, the container includes a coil. In an embodiment, the container houses an electrode. Similarly, the “electrode” is a container, which houses any of the functionalities that are set forth for the “can” container embodiments.

FIG. 1 is an elevation of a pulse generator can according to an embodiment. An electro-medical system 100 is depicted that includes a pulse-generator can 110. The pulse-generator can 110 includes electronics for generating an electrical pulse such as a ventricular contraction signal. In this embodiment, the electro-medical system 100 includes a pulse generator that is a heart pacemaker. In an embodiment, the electro-medical system 100 includes a pulse generator that is an automatic implantable cardioveter defibrillator (AICD), also referred to as a cardio converter. In another embodiment, the electro-medical system 100 includes electronics that are capable of delivering both a ventricular contraction signal and a defibrilator signal. The electro-medical system 100 includes electrical components housed within the can 110 such as a battery, circuitry, and in the embodiment of an AICD, a capacitor.

A porous first covering 112 is located over the can 110. In an embodiment, the porous first covering 112 includes a porous communication to the can. In this embodiment, “porous communication” means when the can 110 has been implanted, an unobstructed, continuous electrical path leads from the outside of the porous first covering 112, to the surface covered by the porous first covering 112. In an embodiment, “porous communication” means an electrical path that exists from living tissue, through the porous first covering 112, to the can 110. The electrical path can be through a medium such as blood plasma, intercellular fluid, and other electrically conductive body humors and tissues.

The electro-medical system 100 can be referred to as a “hot can” where a substantial amount of the surface of the can 110 is capable of electrical communication through the porous first film 112 to living tissue.

In an embodiment, the porous first covering 112 has a pore structure that repels in vivo fibrotic tissue ingrowth. In this embodiment, the porous first covering 112 can act as a wear-minimizing surface. In an embodiment, the pore structure of the porous first covering 112 is smaller than the ordinary cell size of fibrins and other tissues or cells that can grow into implants.

In an embodiment, the porous first covering 112 is an expanded-matrix macromolecule that has an average molecular weight in a range from about 100,000 to about 5,000,000. In an embodiment, the porous first covering 112 is an expanded matrix macromolecule has an average molecular weight in a range from about 800,000 to about 4,000,000. In an embodiment, the porous first covering 112 is an expanded matrix macromolecule that has an average molecular weight in a range from about 100,000 to about 1,000,000. In an embodiment, the porous first covering 112 is an expanded matrix macromolecule that has an average molecular weight in a range from about 1,000,000 to about 3,000,000.

In an embodiment, the porous first covering 112 includes expanded ultra-high molecular weight polyethylene (eUHMWPE). The eUHMWPE can have an expanded matrix that nevertheless repels fibrotic tissue ingrowth. In an embodiment, the porous first covering 112 includes a porous fluropolymer that repels fibrotic tissue ingrowth. In one embodiment, the porous first covering 112 includes a porous poly tetrafluoroethylene (PTFE) that repels fibrotic tissue ingrowth. In an embodiment, the porous first covering 112 includes a porous polyester that repels fibrotic tissue ingrowth. In an embodiment, the porous first covering 112 includes a porous polyurethane that repels fibrotic tissue ingrowth. In an embodiment, the porous first covering 112 includes a porous polyamide that repels fibrotic tissue ingrowth. In an embodiment, the porous first covering 112 includes a combination of at least two of the above compositions.

FIG. 2 is a detail section of the pulse generator can 110 depicted in FIG. 1 that further illustrates an embodiment. FIG. 2 is taken from the section circle 2 in FIG. 1. The can 110 is covered with the porous first covering 112, and a plurality of pores 114 are also illustrated in arbitrary size, shape, and spacing. For example, eUHMWPE or another expanded-matrix molecule can have no straight-though path between an outer surface and an inner surface. In other words, there exists a complex matrix of an expanded macromolecule that both repels in vivo fibrotic tissue ingrowth, and is porous enough to provide an electrical coupling path between a body tissue or fluid and the can 110 if the electrical path to the can 110 is not obstructed with a dielectric coating over that portion of the can 110.

FIG. 3 is an elevation of a pulse generator according to an embodiment. In FIG. 3, an electro-medical system 300 includes a can 310, a porous first covering 312, and a dielectric coating 316. In this embodiment, no electrical path can communicate between living tissue and the can 310. Accordingly, the dielectric coating 316 acts as a substantially complete insulator for the electro-medical system 300. In this embodiment, the porous first covering 312 can act as a wear-minimizing surface. Where the electro-medical system 300 is to be coupled to a lead (not pictured) wear on the lead and the can 310 can be minimized by placing the porous first covering 312 over the dielectric coating 316.

In an embodiment, the dielectric coating 316 is an organic film. In an embodiment, the dielectric coating 316 is a silicone rubber or the like. In an embodiment, the dielectric coating 316 is a polyurethane or the like. In an embodiment, the dielectric coating 316 is a fluoro polymer or the like. In an embodiment, the dielectric coating 316 is a polytetrafluoroethylene (PTFE) or the like. In an embodiment, the dielectric coating 316 is an expanded polytetrafluoroethylene (ePTFE) or the like. In an embodiment, the dielectric coating 316 is a polyolefin or the like. Other embodiments of the dielectric coating 316 include inorganics that are formed as a coating as is known in the art.

In an embodiment, the electro-medical system 300 includes a porous first covering 312 that has a pore structure that repels in vivo fibrotic tissue ingrowth. In an embodiment, the porous first covering 312 is an expanded matrix macromolecule that has an average molecular weight in a range from about 100,000 to about 5,000,000. In another embodiment, the porous first covering 312 is an expanded matrix macromolecule has an average molecular weight in a range from about 800,000 to about 4,000,000. In another embodiment, the porous first covering 312 is an expanded matrix macromolecule that has an average molecular weight in a range from about 100,000 to about 1,000,000. In another embodiment, the porous first covering 312 is an expanded matrix macromolecule that has an average molecular weight in a range from about 1,000,000 to about 3,000,000.

In an embodiment, the porous first covering 312 includes eUHMWPE that repels fibrotic tissue ingrowth. In an embodiment, the porous first covering 312 includes a porous fluropolymer that repels fibrotic tissue ingrowth. In one embodiment, the porous first covering 312 includes a porous PTFE that repels fibrotic tissue ingrowth. In an embodiment, the porous first covering 312 includes a porous polyester that repels fibrotic tissue ingrowth. In an embodiment, the porous first covering 312 includes a porous polyurethane that repels fibrotic tissue ingrowth. In an embodiment, the porous first covering 312 includes a porous polyamide that repels fibrotic tissue ingrowth. In an embodiment, the porous first covering 312 includes a combination of at least two of the above compositions.

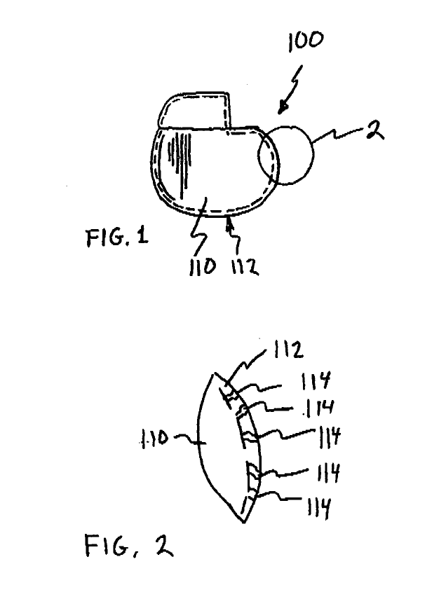

FIG. 4 is an elevation of a pulse generator according to an embodiment. In FIG. 4, an electro-medical system 400 includes a can 410, a porous first covering 412, and a dielectric coating 416. In this embodiment, an electrical path communicates between living tissue and the can 410 because the dielectric coating 416 includes an opening 418 that exposes a portion 419 of the can 410 to an electrical path though the porous first covering 412. Accordingly, the dielectric coating 416 acts as a regional insulator for a part of the can 410, but not total insulator for the electro-medical system 400. In this embodiment, the porous first covering 412 acts both as a wear-minimizing surface, and as an electrical path that communicates between living tissue and the can 410.

Where the electro-medical system 400 is to be coupled to a lead (not pictured) wear on the lead and the can 410 can be minimized by placing the porous first covering 412 over the dielectric coating 416 and over exposed portions of the can 410. In an embodiment, the dielectric coating 416 is an organic film according to the various embodiments set forth herein. In another embodiment, the dielectric coating 416 is an inorganic film as set forth herein.

The porous first covering 412 can be any of the embodiments for the compositions of porous first coverings, including combination embodiments, of the electro-medical systems illustrated in FIGS. 1 and 3.

FIG. 5 is a schematic elevation of a lead according to an embodiment. The lead is an electro-medical system 500 according to an embodiment. The lead includes a proximal end 520, a lead body 522, and a distal end 524 that includes an electrode 526. The electrode 526 includes a porous covering 516 (FIG. 6) that includes a porous communication to the lead. For consistency within this disclosure, the porous covering 516 is referred to as a porous second covering 516. Similar to other embodiments set forth in this disclosure, the porous second covering 516 includes a pore structure that repels in vivo fibrotic tissue ingrowth.

In an embodiment, the electro-medical system 500 includes a porous second covering 516 that is eUHMWPE. Similar to other embodiments set forth in this disclosure, the porous second covering can be of various constructions that include a macro-molecular matrix with the various enumerated ranges of average molecular weights. In other words, the porous second covering 516 can be any of the embodiments for the compositions of porous first coverings, including combination embodiments, of the electro-medical systems illustrated in FIGS. 1, 3, and 4.

FIG. 6 is a detail section of the lead depicted in FIG. 5 that further illustrates an embodiment. FIG. 6 is taken from the section circle 6 in FIG. 5. In an embodiment, the lead body 522 is covered with a porous first covering 512.

The lead is covered with the porous second covering 516, and a plurality of pores 514 are also illustrated in arbitrary size, shape, and spacing. For example, eUHMWPE or another expanded-matrix molecule embodiment can have no straight-though path between an outer surface and an inner surface. In other words, there exists a complex matrix of an expanded macromolecule that both repels in vivo fibrotic tissue ingrowth, and is porous enough to provide an electrical coupling path between a body tissue or fluid and the electrode 526.

A dielectric coating 517 is also partially illustrated that can extend along at least one of the proximal end 520, the lead body 522, and the distal end 524. In the detail section of FIG. 6, a portion of a dielectric coating 517 is illustrated that terminates at the electrode 526. Accordingly, the electrode 526 is only covered with the porous second covering 516. In an embodiment, the porous first covering 512 and the porous second covering 516 are the same composition. In an embodiment, the porous first covering 512 and the porous second covering 516 are different compositions.

FIG. 7 is a cut-away elevation of a lead according to an embodiment. The lead is an electro-medical system 700 according to an embodiment. The lead 700 includes a proximal end 720, a lead body 722, and a distal end 724 that includes a coil 730. The coil 730 includes a porous covering 716 (FIG. 8) that includes a porous communication to the lead 700. For consistency within this disclosure, the porous covering 716 is referred to as a porous second covering 716. Similar to other embodiments set forth in this disclosure, the porous second covering 716 includes a pore structure that repels in vivo fibrotic tissue ingrowth.

In an embodiment, the electro-medical system 700 includes a coil 730 that is used as a defibrilator electrode. In an embodiment, the coil 730 is used as a pacemaker electrode. In an embodiment, the porous covering 716 is disposed over the coil 730 and terminates near the coil upon the distal end 724. Similar to other embodiments set forth in this disclosure, the porous second covering 716 disposed over the coil 730 includes a pore structure that repels in vivo fibrotic tissue ingrowth.

In an embodiment, the electro-medical system 700 includes a porous second covering 716 that is eUHMWPE. Similar to other embodiments set forth in this disclosure, the porous second covering 716 can be of various constructions that include a macro-molecular matrix with the various enumerated ranges of average molecular weights. In other words, the porous second covering 716 can be any of the embodiments for the compositions of porous first and/or second coverings, including combination embodiments, of the electro-medical systems illustrated in FIGS. 1, 3, 4, and 5.

FIG. 8 is a detail section of the lead depicted in FIG. 7 that further illustrates an embodiment. The lead is covered with the porous second covering 716, and a plurality of pores 714 are also illustrated in arbitrary size, shape, and spacing. For example, eUHMWPE or another expanded-matrix molecule can have no straight-though path between an outer surface and an inner surface. In other words, there exists a complex matrix of an expanded macromolecule that both repels in vivo fibrotic tissue ingrowth, but that is porous enough to provide an electrical coupling path between a body tissue or fluid and the coil 730.

A dielectric coating 717 is also partially illustrated that can extend along at least one of the proximal end 720, the lead body 722, and the distal end 724. In the detail section of FIG. 8, a portion of a dielectric coating 717 is illustrated that terminates at the coil 730. Accordingly, the coil 730 is only covered with the porous second covering 716.

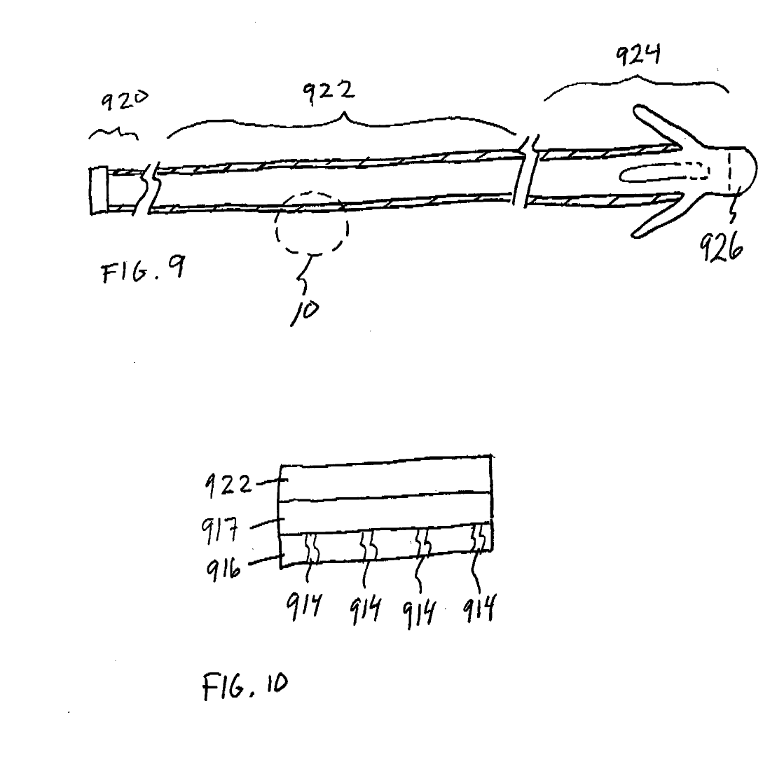

FIG. 9 is a side cross-section of a lead according to an embodiment. The lead is an electro-medical system 900 according to an embodiment. The lead includes a proximal end 920, a lead body 922, and a distal end 924 that includes an electrode 926. The electrode 926 includes a porous covering 916 (FIG. 10) that includes a porous communication to the lead. For consistency within this disclosure, the porous covering 916 is referred to as a porous second covering 916. Similar to other embodiments set forth in this disclosure, the porous second covering 916 includes a pore structure that repels in vivo fibrotic tissue ingrowth.

In an embodiment, the electro-medical system 900 includes the porous second covering 916 that extends the entire length of the lead body 922. Similar to other embodiments set forth in this disclosure, the porous second covering 916 disposed over the electrode 926 includes a pore structure that repels in vivo fibrotic tissue ingrowth.

In an embodiment, the electro-medical system 900 includes a porous second covering 916 that is eUHMWPE. Similar to other embodiments set forth in this disclosure, the porous second covering 916 can be of various constructions that include a macro-molecular matrix with the various enumerated ranges of average molecular weights. In other words, the porous second covering 916 can be any of the embodiments for the compositions of porous first and/or second coverings, including combination embodiments, of the electro-medical systems illustrated in FIGS. 1, 3, 4, 5, and 7.

FIG. 10 is a detail section of the lead depicted in FIG. 9 that further illustrates an embodiment. The lead is covered with the porous second covering 916, and a plurality of pores 914 are also illustrated in arbitrary size, shape, and spacing. For example, eUHMWPE or another expanded-matrix molecule can have no straight-though path between an outer surface and an inner surface. In other words, there exists a complex matrix of an expanded macromolecule that repels in vivo fibrotic tissue ingrowth, but that is porous enough to provide an electrical coupling path between a body tissue or fluid and the electrode 926. A dielectric coating 917 is also partially illustrated that can extend along at least one of the proximal end 920, the lead body 922, and the distal end 924. In the detail section of FIG. 10, the dielectric coating 917 is illustrated as continuous along the length of the lead body 922.

In another embodiment, a combination of a coil and a continuous porous covering is disclosed. In this embodiment, the coil (which is illustrated by way of non-limiting example in FIGS. 7 and 8) and the porous covering that is continuous along the length of the lead body (which is illustrated by way of non-limiting example in FIGS. 9 and 10) are combined. In other words, the porous covering extends substantially the entire length of the lead (e.g., porous covering 916 in FIG. 9), but a breach in the dielectric coating (e.g., dielectric 717 in FIG. 7) occurs at a region along the lead to allow for electrical communication through the porous covering to the coil. In this combination embodiment of a coil and a continuous porous covering, the electro-medical system includes a porous second covering that is eUHMWPE. Similar to other embodiments set forth in this disclosure, the porous second covering can be of various constructions that include a macro-molecular matrix with the various enumerated ranges of average molecular weights. In other words, the porous second covering can be any of the embodiments for the compositions of porous first and/or second coverings, including combination embodiments, of the electro-medical systems illustrated in FIGS. 1, 3, 4, 5, 7, and 9.

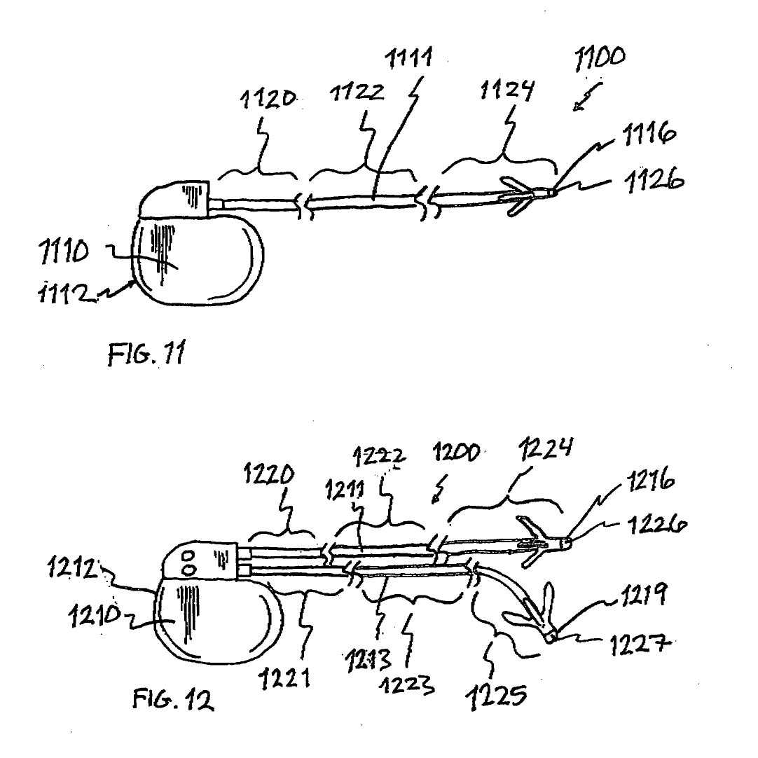

FIG. 11 is an elevation of a pulse generator can and a lead assembly according to an embodiment. An electro-medical system 1100 is depicted that includes a pulse-generator can 1110 and a lead 1111. In an embodiment, the pulse-generator can 1110 includes electronics for generating a pulse such as a ventricular contraction signal. The lead 1111 includes a proximal end 1120, a lead body 1122, and a distal end 1124 that includes an electrode 1126. A porous first covering 1112 is located over the can 1110. The electrode 1126 includes a porous second covering 116 that includes a porous electrical communication to the lead 1111. Similar to other embodiments set forth in this disclosure, the porous second covering 116 includes a pore structure that repels in vivo fibrotic tissue ingrowth.

In an embodiment, the electro-medical system 1100 includes a pulse generator that is a heart pacemaker. In an embodiment, the electro-medical system 1100 includes a pulse generator that is an AICD. In another embodiment, the electro-medical system 1100 includes electronics that are capable of delivering both a ventricular contraction signal and a defibrillator signal.

In an embodiment, the porous second covering 1116 and the porous first covering 1112 are of the same material. In an embodiment, the porous first covering 1112 includes an electrical coupling between the can 1110 and a body tissue or a body fluid as set forth herein. The electro-medical system 1100 can be referred to as a “hot can” system where a substantial amount of the surface of the can 1110 is capable of electrical communication through the porous first covering 1112 to living tissue. In an embodiment, the porous second covering 1116 includes an electrical coupling between the lead 1111 and a body tissue or a body fluid as set forth herein. In another embodiment, the porous first covering 1112 includes a physical communication between a dielectric coating (not pictured) and a body tissue or a body fluid as set forth other embodiments herein.

In an embodiment, at least one of the porous first covering 1112 and the porous second covering 1116 has a pore structure that repels in vivo fibrotic tissue ingrowth. In an embodiment, at least one of the porous first covering 1112 and the porous second covering 1116 has an average pore size that is smaller than the ordinary cell size of fibrins and other tissues or cells that can grow into implants.

In this embodiment, the electro-medical system includes a porous second covering 1116 that is eUHMWPE. Similar to other embodiments set forth in this disclosure, the porous second covering 1116 can be of various constructions that include a macro-molecular matrix with the various enumerated ranges of average molecular weights. In other words, the porous second covering 1116 can be any of the embodiments for the compositions of porous first and/or second coverings, including combination embodiments, of the electro-medical systems illustrated in FIGS. 1, 3, 4, 5, 7, and 9.

FIG. 12 is an elevation of a pulse generator can and a lead assembly according to an embodiment. An electro-medical system 1200 is depicted that includes a pulse-generator can 1210 and a plurality of leads. FIG. 12 illustrates a signal-out first sublead 1211, and a signal-return second sublead 1213. In an embodiment, the plurality of leads is more than two. In an embodiment, the plurality of leads is more than three. In an embodiment, the plurality of leads is more than four. The pulse-generator can 1210 includes electronics for generating a pulse such as a ventricular contraction signal. The pulse-generator can 1210 also includes a porous first covering 1212 located over the can 1210.

The signal-out first sublead 1211, includes a proximal end 1220, a lead body 1222, and a distal end 1224 that includes an electrode 1226. Similarly, the signal-return second sublead 1213, includes a proximal end 1221, a lead body 1223, and a distal end 1225 that includes an electrode 1227.

The electrodes 1226 and 1227 include a porous covering 1216 and 1219, respectively, which include an electrical coupling between the leads 1211 and 1213 and respective local body tissues or fluids etc. In this embodiment, the porous covering 1216 is referred to as a porous second covering 1216, and the porous covering 1219 is referred to as a porous third covering 1217. Similar to other embodiments set forth in this disclosure, the porous second covering 1216 and the porous third covering 1219 includes a pore structure that repels in vivo fibrotic tissue ingrowth.

In an embodiment, the electro-medical system 1200 includes a pulse generator that is a heart pacemaker. In an embodiment, the electro-medical system 1200 includes a pulse generator that is an AICD. In another embodiment, the electro-medical system 1200 includes electronics that are capable of delivering both a ventricular contraction signal and a defibrillator signal.

In an embodiment similar to that depicted in FIG. 7, the porous first covering 1212 is located over the can 1210. In an embodiment, the porous second covering 1216, the porous third covering 1219, and the porous first covering 1212 can be of the same material. In an embodiment, the porous first covering 1212 provides for an electrical coupling between the can 1210 and a body tissue or a body fluid as set forth herein. The electro-medical system 1200 can be referred to as a “hot can” system where a substantial amount of the surface of the can 1210 is capable of electrical communication through the porous first covering 1212 to body tissue or body fluids.

In an embodiment, at least one of the porous first covering 1212, the porous second covering 1216, and the porous third covering 1219 has a pore structure that repels in vivo fibrotic tissue ingrowth. In an embodiment, the pore structure of at least one of the porous first covering 1212, the porous second covering 1216, and the porous third covering 1219 is smaller than the ordinary cell size of fibrins and other tissues or cells that can grow into implants.

In an embodiment, at least one of the porous first covering 1212, the porous second covering 1216, and the porous third covering 1219 includes eUHMWPE. Similar to other embodiments set forth in this disclosure, the porous second covering 1116 can be of various constructions that include a macro-molecular matrix with the various enumerated ranges of average molecular weights. In other words, the porous second covering 1116 can be any of the embodiments for the compositions of porous first and/or second coverings, including combination embodiments, of the electro-medical systems illustrated in FIGS. 1, 3, 4, 5, 7, 9, and 11.

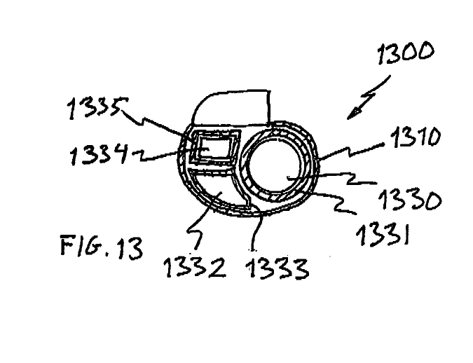

FIG. 13 is a cut-away elevation of a pulse generator can according to an embodiment. In this embodiment a pulse generator 1300 includes a can 1310 and various components within the can 1310.

In FIG. 13, the can 1310 includes a battery 1330, circuitry 1332 in the form of a circuit board, and a capacitor 1334. The size, shape, location, and relative position of each component with respect to the other components, are provided in an arbitrary fashion for illustrative purposes.

In an embodiment, the battery 1330 is enveloped within an eUHMWPE battery closure 1331. In an embodiment, the circuitry 1332 is enveloped within an eUHMWPE circuitry closure 1333. In an embodiment, the capacitor 1334 is enveloped within an eUHMWPE capacitor closure 1335. In each closure embodiment, the closure can be individualized as a separate closure for each component. In another embodiment, each component is inclosed in part of an integral closure composite that provides compartments within the eUHMWPE closure material for each component. In another embodiment, at least one of the components is individually enveloped in an eUHMWPE closure.

In another embodiment, the pulse generator 1300 depicted in FIG. 13 is combined with any other embodiment set forth in this disclosure including any of FIGS. 1-12 and the text supporting those illustrated embodiments. For example, any of the porous coverings can be of various constructions that include a macro-molecular matrix with the various ranges of average molecular weights that are enumerated and set forth in this disclosure. In other words, any or all of the porous coverings can be any of the embodiments for the compositions of porous first, second, and/or third coverings, including combination embodiments, of the electro-medical systems illustrated in FIGS. 1, 3, 4, 5, 7, 9, and 11. Further to an embodiment in FIG. 13, any of the porous macro-molecular matrixes with the various ranges of average molecular weights that are enumerated and set forth in this disclosure, are applicable to any or all of the closure structures, such as the battery closure 1331, the circuitry closure 1333, and the capacitor closure 1335.

The Abstract is provided to comply with 37 C.F.R. §1.72(b) requiring an Abstract that will allow the reader to quickly ascertain the nature and gist of the technical disclosure. It is submitted with the understanding that it will not be used to interpret or limit the scope or meaning of the claims.

In the foregoing Detailed Description, various features are grouped together in a single embodiment for the purpose of streamlining the disclosure. This method of disclosure is not to be interpreted as reflecting an intention that the claimed embodiments of the invention require more features than are expressly recited in each claim. Rather, as the following claims reflect, inventive subject matter lies in less than all features of a single disclosed embodiment. Thus the following claims are hereby incorporated into the Detailed Description, with each claim standing on its own as a separate embodiment

While various embodiments have been described and illustrated with respect to forming buried digit line structures, it should be apparent that the same processing techniques can be used to form other structures by the stacked film techniques set forth in this disclosure for other applications. Furthermore, the processes described herein may be used in the development of other three-dimensional semiconductor structures, as well as in the development of other semiconductor structures, such as gates, interconnects, contact pads, and more.

Although specific embodiments have been illustrated and described herein, it will be appreciated by those of ordinary skill in the art that any arrangement, which is calculated to achieve the same purpose, may be substituted for the specific embodiments shown. Many adaptations of the invention will be apparent to those of ordinary skill in the art. Accordingly, this application is intended to cover any adaptations or variations of the disclosed embodiments.

1. An electro-medical system comprising:
a container including an electrical device therein;
a porous first covering over the container, wherein the porous first covering includes a porous communication to the container.

2. The electro-medical system of claim 1, wherein the porous first covering is selected from expanded ultra-high molecular weight polyethylene, a porous fluropolymer, porous poly tetrafluoroethylene, a porous polyester, a porous polyurethane, a porous polyamide, and combinations thereof.

3. The electro-medical system of claim 1, the system further including:
a lead including a proximal end that is coupled to the container, a lead body, and a distal end including an electrode, wherein the electrode is covered with a porous second covering.

4. The electro-medical system of claim 1, wherein container is completely covered in the porous first covering.

5. The electro-medical system of claim 1, the system further including:
a lead including a proximal end that is coupled to the container, a lead body, and a distal end including a coil, wherein the coil is covered with a porous second covering.

6. The electro-medical system of claim 1, the system further including:
a lead including a proximal end that is coupled to the container, a lead body, and a distal end, wherein at least two of the proximal end, the lead body, and the distal end are covered with a porous second covering.

7. The electro-medical system of claim 1, the system further including:
a lead including a proximal end that is coupled to the container, and a distal end including an electrode, wherein the electrode is covered with a porous second covering; and
wherein at least one of the porous first covering and the porous second covering includes a pore structure that repels in vivo fibrotic tissue ingrowth.

8. The electro-medical system of claim 1, the system further including:
a lead including a proximal end that is coupled to the container, and a distal end including an electrode;
a dielectric coating over the proximal end; and
a porous second covering over the electrode.

9. The electro-medical system of claim 8, wherein the dielectric coating is selected from inorganics, silicone rubber, polyurethane, polytetrafluoro ethylene, fluoro polymers, and polyolefins.

10. The electro-medical system of claim 1, wherein the system further includes a plurality of leads.

11. The electro-medical system of claim 1, the system further including:
a lead including a proximal end that is coupled to the container, and a distal end including an electrode, wherein the electrode is covered with a porous second covering, and wherein the porous second covering is selected from expanded ultra-high molecular weight polyethylene, a porous fluropolymer, a porous poly tetrafluoroethylene, a porous polyester, a porous polyurethane, a porous polyamide, and combinations thereof.

12. The electro-medical system of claim 1, wherein the container houses an electrical device, selected from a cardiac pacemaker, a cardiac defibrillator, a neurostimulator, and a combination thereof.

13. The electro-medical system of claim 1, wherein the container houses a monitor.

14. The electro-medical system of claim 1, wherein the container houses a monitor with a functionality selected from blood pressure, temperature, oxygen, at least one blood sugar, at least one lipoprotein, at least one blood gas, insulin, at least one electrolyte, heart rate, respiration, and a combination of at least two thereof.

15. The electro-medical system of claim 1, wherein the porous first covering over the container is disposed over a dielectric coating, and wherein the dielectric coating causes the container to be one selected from an insulated container and a hot can.

16. An electro-medical system comprising:
a lead including a lead proximal end, a lead body, and a distal end including electrical communication selected from an electrode, a wire, and a coil, wherein lead includes a porous covering that includes a porous communication to the lead, and wherein the porous covering includes a pore structure that repels in vivo fibrotic tissue ingrowth.

17. The electro-medical system of claim 16, wherein the porous covering is selected from expanded ultra-high molecular weight polyethylene, a porous fluropolymer, porous poly tetrafluoroethylene, a porous polyester, a porous polyurethane, a porous polyamide, and combinations thereof.

18. The electro-medical system of claim 16, the system further including:
a container that is coupled to the lead, wherein the container is covered with a porous first covering, and wherein the porous covering on the lead is a porous second covering.

19. The electro-medical system of claim 16, the system further including:
a dielectric coating over at least one of the proximal end and the lead body.

20. The electro-medical system of claim 19, wherein the dielectric coating is selected from inorganics, silicone rubber, polyurethane, polytetrafluoro ethylene, fluoro polymers, and polyolefins.

21. The electro-medical system of claim 16, wherein the lead is one of a plurality of leads.

22. An electro-medical system, comprising:
a container including an electrical device;
a dielectric coating over the container;
a passage through the dielectric coating to form an exposed portion of the container; and
a porous first covering over the exposed portion of the container.

23. The electro-medical system of claim 22, wherein the porous first covering is selected from expanded ultra-high molecular weight polyethylene, a porous fluropolymer, porous poly tetrafluoroethylene, a porous polyester, a porous polyurethane, a porous polyamide, and combinations thereof.

24. The electro-medical system of claim 22, the system further including:
a lead including a proximal end that is coupled to the container, a lead body, and a distal end including an electrode, wherein the electrode is covered with a porous second covering.

25. The electro-medical system of claim 22, the system further including:
a lead including a proximal end that is coupled to the container, a lead body, and a distal end including an electrode, wherein at least two of the proximal end, the lead body, the distal end, and the electrode are covered with a porous second covering.

26. The electro-medical system of claim 22, the system further including:
a lead including a proximal end that is coupled to the container, and a distal end including an electrode, wherein the electrode is covered with a porous second covering; and
wherein at least one of the porous first covering and the porous second covering has a pore structure that repels in vivo fibrotic tissue ingrowth.
